(19) United States
(12) Patent Application Publication
(10) Pub. No.: US 2006/0258635 A1
(43) Pub. Date: Nov. 16, 2006

(54) Use of a porphyrin compound for the treatment of skin fungi

(75) Inventors: Geertrudia Maria Theresia Smijs, Leiden (NL); Johannes Joseph Schuitmaker, Leiden (NL)

Correspondence Address: AKERMAN SENTERFITT, P.O. BOX 3188, WEST PALM BEACH, FL, 33402-3188, US

(73) Assignee: PHOTOBIOCHEM N.V., Al Leiden (NL)

(21) Appl. No.: 10/544,813

(22) PCT Filed: Feb. 5, 2004
(86) PCT No.: PCT/NL04/00079

§ 371 Date: Apr. 7, 2006

(30) Foreign Application Priority Data
Feb. 5, 2003 (NL) 1022597

(51) Int. Cl. A61K 31/444; A61K 31/409; A61K 31/555

(52) U.S. Cl. 514/185; 514/332; 514/410

(57) ABSTRACT

The present invention relates to the use of a photosensitizer compound with a specified structural formula for the treatment of a skin-borne fungus, a method of preparing a pharmaceutical composition for the treatment of a skin-borne fungus, and a method of treating a mammal suffering from a skin-borne fungus, such as Trichophyton rubrum.

The present invention relates to a use of a photosensitizer compound for the preparation of a pharmaceutical composition.

According to the present invention, the use concerns the use of a photosensitizer compound chosen from the group consisting of compounds with the formulas Ia-Id 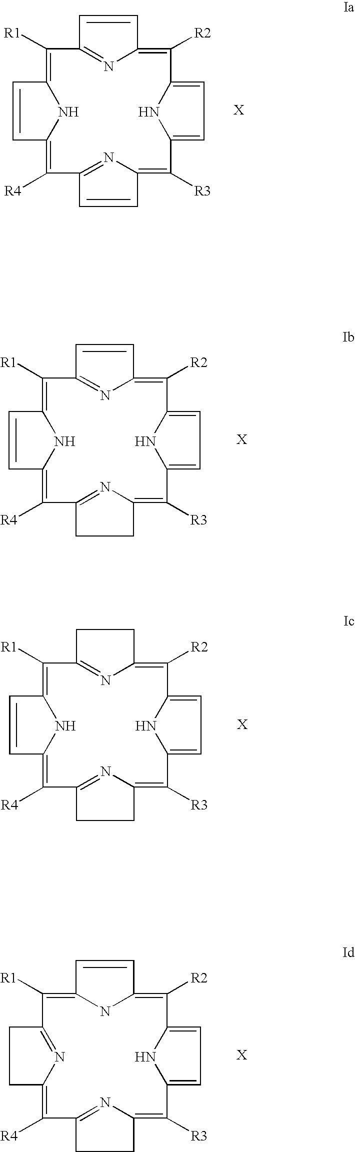
wherein R₁, R₂, R₃ and R₄ are independently chosen from the group consisting of
hydrogen,
a halogen atom,
(C₁-C₂₀)alkyl, (C₃-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which is optionally substituted with one or more groups chosen from
   hydroxyl,
   amino which is optionally substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these is optionally substituted with one or more groups chosen from hydroxyl, and a halogen atom,
   nitril, and
   a halogen atom,
(C₆-C₂₀)aryl, and (C₆-C₂₀)heterocyclic aryl group each of which is optionally substituted with one or more groups chosen from
   hydroxyl,
   amino which is optionally substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom,
   nitril,
   25 a halogen atom, and
   (C₁-C₁₀)alkyl, (C₁-C₁₀)alkoxy, (C₂-C₁₀)alkenyl, the heterocyclic aryl group containing at least one atom chosen from N, O, P, and S where P, N or S is optionally substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom,
at least one of the groups R₁, R₂, R₃ and R₄ contains a quaternary nitrogen atom, and
wherein X is a pharmaceutically acceptable counterion for the manufacture of a pharmaceutical composition for the treatment of skin-borne fungus.

Preferably, at least one of R₁, R₂, R₃ and R₄ is a (C₆-C₂₀)heterocyclic aryl group comprising a nitrogen atom substituted with a group chosen from (C₁-C₂₀)alkyl, (C₂-C₂₀)alkenyl, (C₁-C₁₀)alkoxy, and (C₂-C₂₀)alkynyl, each of which may be linear or branched and each of which is optionally substituted with one or more groups chosen from hydroxyl, and a halogen atom.

Advantageously, the heterocyclic aryl group is a pyridinium group, the nitrogen of which is substituted with a (C₁-C₄)alkyl group.

According to a preferred embodiment, at least one of R₁, R₂, R₃ and R₄ is a (C₆-C₂₀)aryl group substituted with an amino which is optionally substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom.

According to a preferred embodiment, the aryl group is a trialkyl aminophenyl group where each alkyl independently is (C₁-C₃)alkyl.

For the method according to the invention, it is preferred that at least two of R₁, R₂, R₃ and R₄ comprise a quaternary nitrogen atom.

More preferably, three of R₁, R₂, R₃ and R₄ comprise a quaternary nitrogen atom.

Most preferably, the photosensitizer compound is mono-phenyl-tri(N-methyl-4-pyridyl)porphyrin chloride (Sylsens B).

All the above embodiments result in a more effective pharmaceutical composition for the treatment of skin-borne fungal diseases. This may result in the use of a reduced amount of active compound (photosensitizer compound), which saves cost, or in a less time-consuming treatment.

According to an important embodiment, the use concerns the preparation of a topical composition, i.e. involving combining the photosensitizer compound with a pharmaceutical excipient for topical application. Such an excipient is for example a gel, lotion or an ointment.

The invention also relates to a method of preparing a pharmaceutical composition for the treatment of skin-borne fungus comprising combining a compound chosen from the group consisting of compounds with the formulas Ia-Id 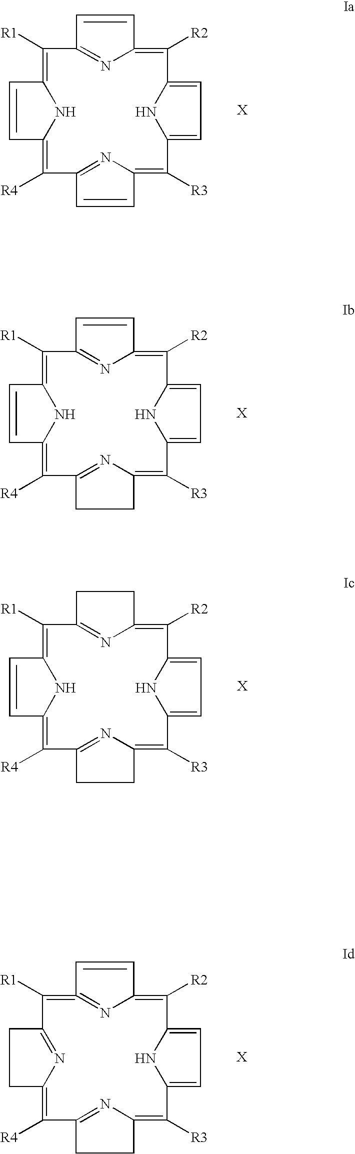
wherein R₁, R₂, R₃ and R₄ are independently chosen from the group consisting of
hydrogen,
a halogen atom,
(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which is optionally substituted with one or more groups chosen from
   hydroxyl,
   amino which is optionally substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these is optionally substituted with one or more groups chosen from hydroxyl, and a halogen atom,
   nitril, and
   a halogen atom,
(C₆-C₂₀)aryl, and (C6-C20)heterocyclic aryl group each of which is optionally substituted with one or more groups chosen from
   hydroxyl,
   amino which is optionally substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom,
   nitril,
   a halogen atom, and
   (C₁-C₁₀)alkyl, (C₁-C₁₀)alkoxy, (C₂-C₁₀)alkenyl,
the heterocyclic aryl group containing at least one atom chosen from N, O, P, and S where P, N or S are optionally substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl and a halogen atom,
at least one of the groups R₁, R₂, R₃ and R₄ contains a quaternary nitrogen atom, and
wherein X is a pharmaceutically acceptable counterion and a pharmaceutically acceptable carrier or excipient.

Preferably, a substance is added capable of making skin and/or nail more permeable to the compound.

This allows for a more effective and less time-consuming treatment.

According to an important embodiment, the pharmaceutical composition is a topical composition.

Finally, the invention relates to a method of treating a mammal, said treatment comprising the application of a pharmaceutical composition comprising a compound chosen from the group consisting of compounds with the formulas Ia-Id and illuminating the location where the pharmaceutical composition is applied with light having a wavelength which can be absorbed by the compound in the presence of oxygen.

It is not necessary to add oxygen, as this is readily available in the atmosphere and the tissue being treated.

Preferably, light is used with a wavelength near or at an absorption maximum of the compound.

The present invention will now be illustrated by way of example, in particular by way of the preferred embodiment, with reference to the drawing where

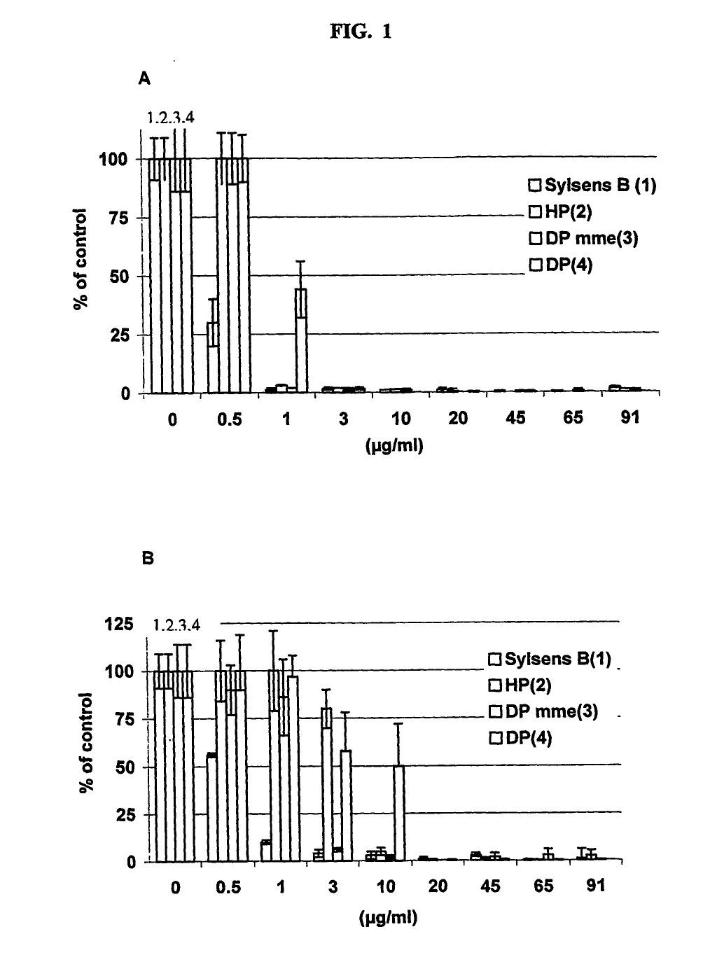

FIGS. 1a and 1b show the effectiveness of various compounds in suppressing the growth of a dermatophyte by photodynamic treatment; and

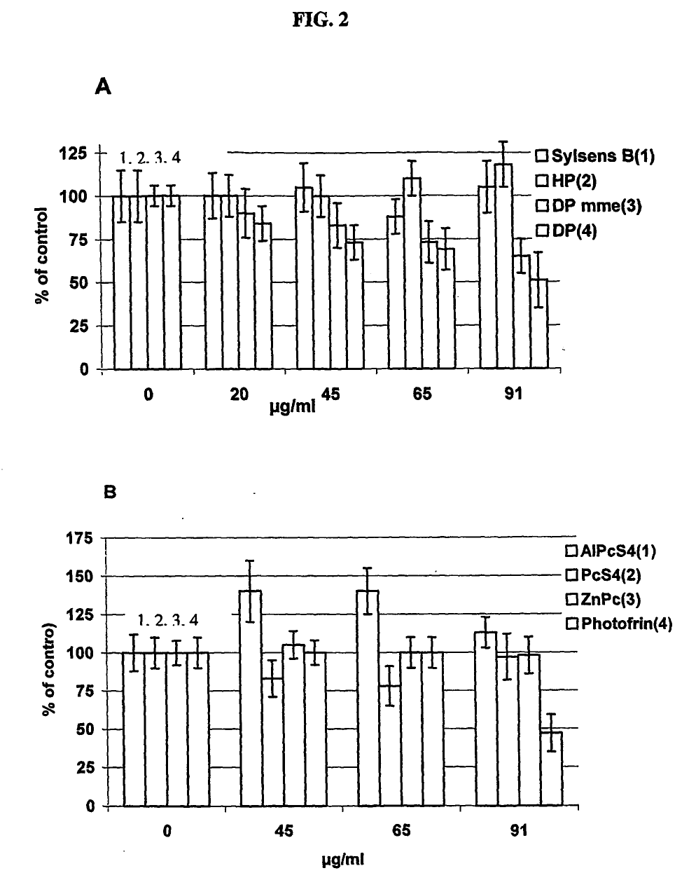

FIG. 2 is similar to FIG. 1 and displays the data obtained for reference photosensitizer compounds

### MATERIALS AND METHODS

Materials The fungus Trichophyton rubrum was purchased from the Centraalbureau voor Schimmelcultures (CBS), Baarn, The Netherlands. Cultures were grown on Malt Extract Agar (MEA, Oxoid, Hampshire England). Suspension cultures were made in Dulbecco's Modified Eagle Medium (DMEM, GibcoBRL, UK) with 2.5 % Fetal Calf Serum (FCS, GibcoBRL, UK).

Solid cultures were maintained at 28° C., the suspension cultures at room temperature. Hematoporphyrin (HP) was purchased from Porphyrin Products Inc. (Utah, USA), 5,10,15-tris(4-methylpyridinium)-20-phenyl-[21H, 23H]-porphyrine trichloride (Sylsens B), deuteroporphyrin (DP) and deuteroporphyrin monomethylester (DP mme) were synthesized and kindly provided by the Department of Bio-Organic Photochemistry, Leiden University, the Netherlands (purity, checked with NMR was more than 99.5%). All the phthalocyanines were purchased from Porphyrin Products. Inc. (Utah, USA)) and Photofrin was purchased from Lederle Parenterals Inc. (Carolina, USA). Polyethylene-glycol was obtained from Genfarma B.V. (Maarssen, the Netherlands), while all other chemicals were purchased from J.T.Baker (Deventer, The Netherlands).

The following solvents were used:
50 mM sodium phosphate buffer pH 7.4 for Sylsens B, HP, Photofrin, ZnPc and AlPcS4;
polyethyleneglycol:ethanol:water (3:2:5) for DP and DP mme; and
dimethylformamide (DMF) for PcS4.

Stock solutions of the photosensitizers of 2.5 mg/ml solvent were stored at 40° C. for no longer than one week.

Photodynamic treatment Illuminations were performed with a lamp from “MASSIVE” (no.74900/21), 1× max. 500W-230 V-R7s, IP 44. To avoid heating of the samples to be illuminated, the white light produced by the lamp first passes a 2 cm thick water layer before reaching the samples. The light intensity was measured with a IL1400A photometer equipped with a SEL033/F/U detector (International Light, Newburyport, Mass., USA). Before illumination, the fungal culture suspensions were incubated with the photosensitizer in test tubes for 30 minutes at a temperature of 28° C. After incubation, the suspension cultures were illuminated in the presence of the sensitizer in 3 cm diameter culture dishes (Greiner, Alphen aan den Rijn, The Netherlands). After illumination, the contents of the culture dishes were transferred to dishes of 9 cm diameter containing MEA, placed in the incubator at 28° C. and growth was monitored during one week and quantified by counting the number of inoculates present.

Results

Photodynamic Treatment of Trichophyton rubrum

Photodynamic treatment of Trichophyton rubrum in suspension culture by Sylsens B and DP mme resulted in completely killing the fungus in almost all experiments. Of the porphyrin derivatives Sylsens B is by far the most effective. As can be seen from FIG. 1A, the photodynamic efficacy of Sylsens B and DP mme above a concentration of 3 μg/ml is analogous to the effect caused by the other porphyrins, HP and DP, that were tested. Below this concentration, Sylsens B displayed a better photodynamic efficacy, while the efficacy of DP mme was the same as detected with HP; DP gave the poorest efficacy in this lower concentration range. Percentages on the Y-axis are percentages where the control is 100% (no photosensitizer added). Compared to FIG. 1B it can be seen that at lower sensitizer concentrations PDT merely results in a delay in growth. Only above a concentration of 20 μg/ml was a true fungicidal effect detected for all the porphyrin sensitizers tested. Sylsens B and DP mme displayed this effect at an even lower concentration, namely at 3 μg/ml. For all the porphyrins it was established that successful PDT meant that even after several weeks no trace of a recurrence of the fungus could be detected on the MEA dishes. When examining FIG. 2A, however, it becomes clear that DP mme as well as DP expresses a dark toxicity at higher concentrations. Sylsens B, however, and HP show no dark toxicity under the given circumstances.

FIG. 2 shows the result of a photodynamic treatment of Trichophyton rubrum with the use of several phthalocyanines and Photofrin. The observed photodynamic efficacy towards Trichophyton rubrum is not as high as found for the porphyrins (compare to FIG. 1A). In the case of the phthalocyanines or Photofrin, the observed effect is merely a delay of growth in the first days after the photodynamic treatment. After 7 days the fungus grows again as well as it did without photodynamic treatment, displaying a 100 percent survival over the whole concentration range used. This phenomenon was observed not only for PcS4 and ZnPc but for Photofrin and AlPcS4 as well. Treatment with light then gives results that are similar to the results obtained with just solvent instead of the photosensitizer. So these photosensitizers display a fungistatic effect of 7 days. Concerning the dark toxicity, only Photofrin displays a positive result when applied at concentrations from 90 μg/ml and higher (see FIG. 2B). For the phthalocyanines no dark toxicity towards Trichophyton rubrum was observed under the given circumstances.

In view of the above results, it can be concluded that the claimed porphyrins, in contrast to the reference photosensitizer phthalocyanins compounds, provide an excellent fungistatic effect on the dermatophyte T. rubrum.

1. A method of treatment of skin-borne fungal diseases comprising:
administering a pharmaceutical composition including a photosensitizer compound chosen from the group consisting of compounds with the formulas Ia-Id 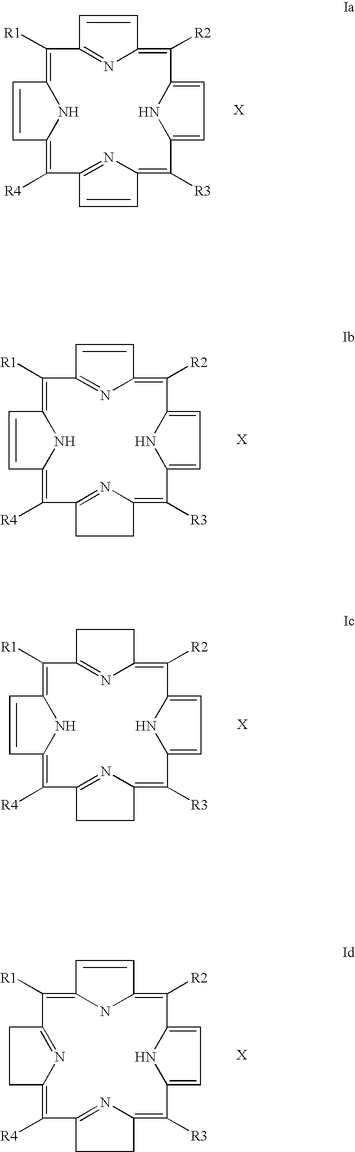
wherein R₁, R₂, R₃ and R₄ are independently chosen from the group consisting of
hydrogen,
a halogen atom,
(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which is optionally substituted with one or more groups chosen from
hydroxyl,
amino which is optionally substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these is optionally substituted with one or more groups chosen from hydroxyl, and a halogen atom,
nitril, and
a halogen atom,
(C₆-C₂₀)aryl, and (C₆-C₂₀)heterocyclic aryl group, each of which is optionally substituted with one or more groups chosen from
hydroxyl,
amino which is optionally substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom,
nitril,
a halogen atom, and
(C₁-C₁₀)alkyl, (C₁-C₁₀)alkoxy, (C₂-C₁₀)alkenyl,
the heterocyclic aryl group containing at least one atom chosen from N, O, P, and S where P, N or S are optionally substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom,
at least one of the groups R₁, R₂, R₃ and R₄ contains a quaternary nitrogen atom, and
wherein X is a pharmaceutically acceptable counterion.

2. The method according to claim 1, wherein at least one of R₁, R₂, R₃ and R₄ is a (C₆-C₂₀)heterocyclic aryl group comprising a nitrogen atom substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom.

3. The method according to claim 2, wherein the heterocyclic aryl group is a pyridinium group, the nitrogen of which is substituted with a (C₁-C₄)alkyl group.

4. The method according to claim 1, wherein at least one of R₁, R₂, R₃ and R₄ is a (C₆-C₂₀)aryl group substituted with an amino which is optionally substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom.

5. The method according to claim 4, wherein the aryl group is a trialkyl aminophenyl group where each alkyl independently is (C₁-C₃)alkyl.

6. The method according to claim 1, wherein at least two of R₁, R₂, R₃ and R₄ comprise a quaternary nitrogen atom.

7. The method according to claim 6, wherein three of R₁, R₂, R₃ and R₄ comprise a quaternary nitrogen atom.

8. The method according to claim 7, wherein the photosensitizer compound is monophenyl-tri(N-methyl-4-pyridyl)porphyrin chloride.

9. A method of preparing a pharmaceutical composition for the treatment of skin-borne fungus comprising combining a compound chosen from the group consisting of compounds with the formulas Ia-Id 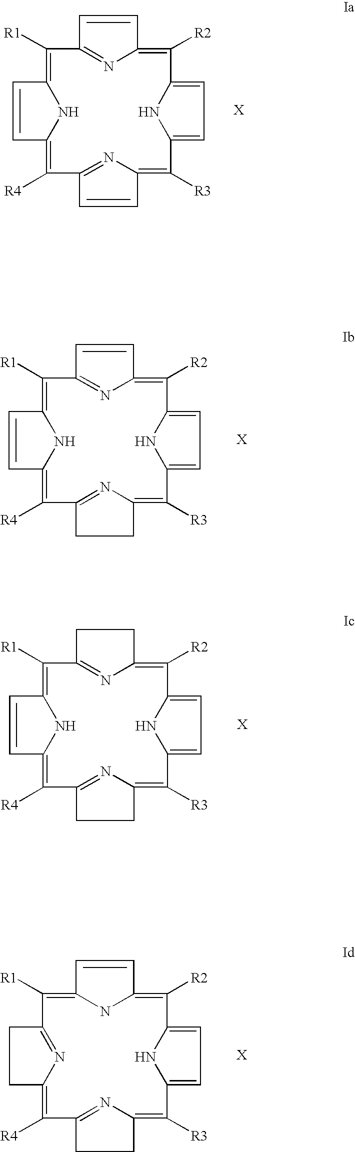
wherein R₁, R₂, R₃ and R₄ are independently chosen from the group consisting of
hydrogen,
a halogen atom,
(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which is optionally substituted with one or more groups chosen from
hydroxyl,
amino which is optionally substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these is optionally substituted with one or more groups chosen from hydroxyl, and a halogen atom,
nitril, and
a halogen atom,
(C₆-C₂₀)aryl, and (C₆-C₂₀)heterocyclic aryl group each of which is optionally substituted with one or more groups chosen from
hydroxyl,
amino which is optionally substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl, and a halogen atom,
nitril,
a halogen atom, and
(C₁-C₁₀)alkyl, (C₁-C₁₀)alkoxy, (C₂-C₁₀)alkenyl,
the heterocyclic aryl group containing at least one atom chosen from N, O, P, and S where P, N or S are optionally substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which is optionally substituted with one or more groups chosen from hydroxyl
and a halogen atom,
at least one of the groups R₁, R₂, R₃ and R₄ contains a quaternary nitrogen atom, and
wherein X is a pharmaceutically acceptable counterion and a pharmaceutically acceptable carrier or excipient.

10. The method according to claim 9, wherein a substance is added capable of making skin and/or nail more permeable to the compound.

11. (canceled)
